(19) United States
(12) Patent Application Publication
(10) Pub. No.: US 2022/0370604 A1
(43) Pub. Date: Nov. 24, 2022

(54) PEPTIDE VACCINE FOR VIRUS INFECTION

(71) Applicant: Vacino Biotech Co., Ltd., Taipei (TW)

(72) Inventors: Jia-Ming CHANG, Taipei (TW); Yi-Ru LEE, Taipei (TW)

(21) Appl. No.: 17/726,688

(22) Filed: Apr. 22, 2022

Related U.S. Application Data
(60) Provisional application No. 63/190,116, filed on May 18, 2021.

(51) Int. Cl. A61K 39/39; A61P 37/04; A61P 31/14; C07K 14/74

(52) U.S. Cl. CPC A61K 39/39; A61P 37/04; A61P 31/14; C07K 14/70539; A61K 2039/5158

(57) ABSTRACT

The present invention relates to an immunogenic composition against virus infection, especially to an immunogenic composition having peptides that are capable of binding to major histocompatibility complex (MHC) molecules and inducing a broad-spectrum immunity against coronavirus.

### CROSS-REFERENCE TO RELATED APPLICATIONS

This application claims priority to and the benefit of U.S. Provisional Application No. 63/190,116, filed on May 18, 2021, the disclosure of which is incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: P22-0076 Sequence Listing ST25.txt, date recorded: Apr. 21, 2022, size: 5 KB).

### BACKGROUND OF THE INVENTION

### 1. Field of the Invention

The present invention relates to an immunogenic composition against virus infection, especially to an immunogenic composition having peptides that are capable of binding to major histocompatibility complex (MHC) molecules and inducing a broad-spectrum immunity against coronavirus.

### 2. Description of the Prior Art

Severe acute respiratory syndrome coronavirus 2 (SARS-CoV-2) is the virus that causes coronavirus disease 2019 (COVID-19), which has been the global pandemic infection since late 2019. SARS-CoV-2 was firstly identified in Wuhan, China, in December 2019, and as of 3 Apr. 2022, 489,779,062 confirmed cases of COVID-19, including 6,152,095 deaths, have been reported to the WHO (https://covid19.who.int). The numbers are still growing fast.

SARS-CoV-2 is persistent in evolution through spreading among people. To date, there are 6 major subtypes of SARS-CoV-2 viruses discovered worldwide. The mutations are occurred in the spike protein of SARS-CoV-2 at the position of P681H (B.1.1.207), N501Y/69-70del/P681H (B.1.1.7, Alpha variant), N501Y/K417N/E484K (B.1.351, Bata variant), N501Y/E484K/K417T (P.1, Gamma variant), ten mutations (B.1.617.2) of Delta and thirty mutations (B.1.1.529) of Omicron. The mutation of SARS-COV-2 virus is continuing and the worse is occurred the hybrid of two different subtypes of COVID-19 viruses, as XD, XE and XF etc. Due to the high mutation rate of SARS-CoV-2, it is urgent to develop a broad spectrum COVID-19 vaccine to stop the COVID-19 pandemic.

### SUMMARY OF THE INVENTION

The present invention relates to peptides designed based on coronavirus spike proteins. The peptides may be used to diagnose, prevent, or treat coronavirus infections in humans. The present inventors have designed number of peptides that are conserved between different coronaviruses and are capable of binding to a molecule of a major histocompatibility complex (MHC). Inclusion of one or more such peptides in a vaccine composition may confer protective capability against one or more coronaviruses, and/or the ability to treat existing coronavirus infection. Each of the peptides may also be used to diagnose the presence or absence of coronavirus infection, for instance by detecting in a sample the presence or absence of a molecule (such as a T cell receptor or antibody) that is capable of binding to the peptide. The coronavirus may, for example, be a coronavirus that is implicated in a human epidemic or pandemic. The coronavirus may, for example, be a coronavirus of zoonotic origin. The coronavirus may, for example, be a member of the genus Betacoronavirus. The coronavirus may, for example, be a member of subgenus Sarbecoronavirus. The coronavirus may, for example, be SARS coronavirus or SARS coronavirus 2.

Accordingly, the present invention provides a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, and wherein the variant binds to a molecule of an MHC and/or induces T cells cross-reacting with the variant peptide.

The present invention also provides:
a nucleic acid encoding the peptides of the present invention;
an immunogenic composition, comprising the peptides of the present invention or the nucleic acid of the present invention;
an antibody specifically recognizing the peptide of the present invention;
a T-cell receptor being capable of binding to the peptide of the present invention.
a recombinant host cell, comprising one component selected from the group consisting of the peptide of the present invention, the nucleic acid of the present invention, the antibody or fragment thereof of the present invention, and the T-cell receptor or fragment thereof of the present invention;
an in vitro or ex vivo method for producing activated T lymphocytes, comprising contacting in vitro or ex vivo T cells with antigen loaded human class I or II MHC molecules expressed on the surface of a suitable antigen-presenting cell or an artificial construct mimicking an antigen-presenting cell for a period of time sufficient to activate the T cells in an antigen specific manner, wherein the antigen is the peptide of the present invention;
an activated T lymphocyte produced by the method mentioned above, wherein the activated T lymphocyte selectively recognizes a cell presenting the peptide of the present invention.
a pharmaceutical composition, comprising at least one active ingredient selected from the group consisting of the peptide of the present invention, the nucleic acid of the present invention, the antibody or fragment thereof of the present invention, the T-cell receptor or fragment thereof of the present invention, the recombinant host cell of the present invention, and the activated T lymphocyte of the present invention;
a method for preventing or treating a pathogenic infection in a subject in need thereof, comprising administering to the subject an effective amount of the immunogenic composition or the pharmaceutical composition of the present invention;
a method for generating anti-coronavirus antibodies, comprising administering the immunogenic composition of the present invention to an animal or a human subject;
a complex, comprising the peptide of the present invention bound to an MHC molecule;
a method for determining the presence or absence of current or previous coronavirus infection in an individual, comprising contacting the peptide or the complex of the present invention with a sample obtained from the individual and determining the presence or absence of binding between the peptide or complex and a molecule comprised in the sample;
a method for identifying coronavirus-specific T cells, comprising contacting the peptide or the complex of the present invention with a sample obtained from an individual and determining the presence or absence of binding between the peptide or complex and a T cell receptor comprised in the sample; and
a method for identifying a coronavirus-specific T cell receptor, comprising contacting the peptide or the complex of the present invention with a T cell receptor and determining the presence or absence of binding between the peptide or complex and the T cell receptor.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows the HLA class I binding ability of Peptide 1 (SEQ ID NO: 1), Peptide 12 (SEQ ID NO: 12) of the present invention, and CMVpp65₄₉₅₋₅₀₃ (SEQ ID NO: 27; positive control) at the concentration of 1 nM, 3 nM, and 8.9 nM. Different concentrations of peptides were incubated with β2-microglobulin light chain subunit and biotin-labeled recombinant HLA-A201 to form peptide-HLA complexes. The peptide-HLA complexes were then captured by streptavidin-coated beads and detected by anti-human β2-microglobulin antibody on flow cytometry. Each data point represents the mean fluorescence intensity corresponding to the number of the formed peptide-HLA complex, and the error bars represent standard deviation (n=2).

FIG. 2 shows the HLA class I binding ability of Peptide 1-26 (SEQ ID NOs: 1-26) of the present invention at the concentration of 8.9 nM. MHC-peptide binding assay was performed as described in FIG. 1. Each bar represents the mean fluorescence intensity corresponding to the number of the formed peptide-HLA complex, and the error bars represent standard deviation (n=2).

FIG. 3 shows the results of MHC class I tetramer assay of Peptide 1 (SEQ ID NO: 1) and Peptide 12 (SEQ ID NO: 12) of the present invention. Human PBMCs were treated with MHC class I tetramers binding with Peptide 1 (SEQ ID NO: 1) or Peptide 12 (SEQ ID NO: 12) for 20 minutes, co-stained with anti-CD8 antibodies, and then analyzed with a flow cytometer. Results are presented as mean with error bars representing standard error (n=3). *p<0.05.

FIG. 4A shows cytokine (IFN-γ and IL-4) release of CD4⁺ T cells in response to Peptide 1 (SEQ ID NO: 1) of the present invention on Day 12 of the peptide treatment. Human PBMCs were treated with Peptide 1 (SEQ ID NO: 1) at the concentrations of 0.4, 2, 10, 50, 250 nM for 12 days and then subject to intracellular cytokine staining and multiparameter flow cytometry. Each data point represents the mean fluorescence intensity corresponding to the amount of cytokine, and the error bars represent standard error (n=3).

FIG. 4B shows cytokine (IFN-γ and IL-4) release of CD4⁺ T cells in response to Peptide 12 (SEQ ID NO: 12) of the present invention on Day 12 of the peptide treatment. Human PBMCs were treated with Peptide 12 (SEQ ID NO: 12) at the concentrations of 0.4, 2, 10, 50, 250 nM for 12 days and then subject to intracellular cytokine staining and multiparameter flow cytometry. Each data point represents the mean fluorescence intensity corresponding to the amount of cytokine, and the error bars represent standard error (n=3).

FIG. 5A shows the total IgG amount in BALB/c mice 2 weeks after each immunization. BALB/c mice (n=10 per group) were intramuscularly injected three times at 2 weeks apart (on Days 0, 14, and 28) with 45 μg of Peptide 1 (SEQ ID NO: 1) of the present invention. Antisera were collected one day prior to the first injection and 2 weeks after each injection (on Days−1, 13, 27, and 41) and subject to total IgG ELISA assays. Each dot represents total IgG amount of each individual serum sample. Each box plot depicts measurements from the 25th to 75th percentile. The error bars correspond to the 10th and 90th percentiles, and the horizontal bar in each box represents the average. ***p<0.001.

FIG. 5B shows the anti-spike IgG titer in BALB/c mice 2 weeks after the third immunization. The mice were immunized as described in FIG. 5A (n=10 per group), and the antisera were collected one day prior to the first injection (on Day−1, pre-dose) and 2 weeks after the third injection (on Day 41, post-dose) and subject to anti-spike IgG ELISA assay. Each dot represents anti-spike IgG titer of each individual serum sample. Each box plot depicts measurements from the 25th to 75^{th} percentile. The error bars correspond to the 10^{th} and 90^{th} percentiles, and the horizontal bar in each box represents the average. ***p<0.001.

FIG. 6A shows the total IgG amount in BALB/c mice 2 weeks after the third immunization. BALB/c mice (n=3-4 per group) were intramuscularly injected three times at 2 weeks apart (on Days 0, 14, and 28) with 400 μg of Peptide 12 (SEQ ID NO: 12) of the present invention. Antisera were collected one day prior to the first injection (on Day−1, pre-dose) and 2 weeks after the third injection (on Day 41, post-dose) and subject to total IgG ELISA assays. Each dot represents total IgG amount of each individual serum sample, and the horizontal bars represent the average of each group. ***p<0.001.

FIG. 6B shows the anti-spike IgG titer in BALB/c mice 2 weeks after the third immunization. The antisera were collected as described in FIG. 6A (n=4 per group) and subject to anti-spike IgG ELISA assay. Each dot represents anti-spike IgG titer of each individual serum sample, and the horizontal bars represent the average of each group. *p<0.05, ***p<0.001.

FIG. 7A shows the total IgG amount in BALB/c mice 2 weeks after the third immunization. BALB/c mice (n=5 per group) were orally administrated three times at 2 weeks apart (on Days 0, 14, and 28) with 200 μg of Peptide 1 (SEQ ID NO: 1) of the present invention formulated with 1% (v/v) of PLCL-PEG-PLCL. Antisera were collected one day prior to the first administration (on Day−1, pre-dose) and 2 weeks after the third injection (on Day 41, post-dose) and subject to total IgG ELISA assays. Each dot represents total IgG amount of each individual serum sample, and the horizontal bars represent the average of each group. ***p<0.001.

FIG. 7B shows the anti-spike IgG titer in BALB/c mice 2 weeks after the third immunization. The antisera were collected as described in FIG. 7A (n=5 per group) and subject to anti-spike IgG ELISA assay. Each dot represents anti-spike IgG titer of each individual serum sample, and the horizontal bars represent the average of each group. **p<0.01.

### DETAILED DESCRIPTION OF THE PREFERRED EMBODIMENT

The present invention relates to a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, and wherein the variant binds to a molecule of a major histocompatibility complex (MHC) and/or induces T cells cross-reacting with the variant peptide. SEQ ID NOs: 1 to 26 are set out in Table 1.

TABLE 1

| SEQ ID NO | Sequence |
|---|---|
| 1 | KLNSGDSKV |
| 2 | KMNSGDSKV |
| 3 | KINSGDSKV |
| 4 | KQNSGDSKV |
| 5 | KVNSGDSKV |
| 6 | KLNSGDSFV |
| 7 | KLNSGDSKL |
| 8 | KLNSGDSKI |
| 9 | KLNSGDSKA |
| 10 | KLNSGDSPV |
| 11 | KLNSGISKV |
| 12 | KLNSGLSKV |
| 13 | KLNSGVSKV |
| 14 | KLMSGDSKV |
| 15 | KLWSGDSKV |
| 16 | KLFSGDSKV |
| 17 | KLYSGDSKV |
| 18 | FLNSGDSKV |
| 19 | YLNSGDSKV |
| 20 | KLNSGDWKV |
| 21 | KLNSGDFKV |
| 22 | KLNSGDYKV |
| 23 | KLNSGDSYV |
| 24 | KLNSGDSKVV |
| 25 | KLNSGDSKVL |
| 26 | FKLNSGDSKV |

To design SEQ ID NOs: 1 to 26, well-conserved regions of human SARS-CoV-2 spike proteins were selected, and short antigenic peptides with 9 to 10 residues long were generated based on human leukocyte antigen (HLA) molecule structure. These peptides were then subject to HLA binding assay to confirm their binding activity to HLA molecule, in which peptides having strong binding activity to HLA were selected. Therefore, the peptides of the invention are capable of binding to an MHC molecule, especially in an MHC class I molecule.

In some embodiments, the peptides of the present invention have the ability to bind to an MHC class-I or II molecule, and when the peptides are bound to the MHC, the peptides are capable of being recognized by CD4⁺ and/or CD8⁺ T cells.

In some embodiments, the variant sequences are at least 80% homologous to SEQ ID NO: 1 to SEQ ID NO: 26. In some embodiments, the variant sequences are at least 80%, at least 85%, at least 88%, at least 90%, at least 95% homologous to SEQ ID NO: 1 to SEQ ID NO: 26. Each possibility represents a separate embodiment of the invention.

As used herein, the terms “coronavirus (CoV)” refers to a group of related RNA viruses of the family Coronaviridae that cause diseases in mammals and birds. Seven human coronaviruses (HCoVs) have been so far identified, namely HCoV-229E, HCoV-OC43, HCoV-NL63, HCoV-HKU1, severe acute respiratory syndrome coronavirus (SARS-CoV), Middle East respiratory syndrome coronavirus (MERS-CoV) and the novel coronavirus (2019-nCoV, a.k.a. SARS-CoV-2). Unlike the highly pathogenic SARS-CoV, MERS-CoV, and 2019-nCoV, the four so-called common HCoVs generally cause mild upper-respiratory tract illness and contribute to 15% to 30% of cases of common colds in human adults, although severe and life-threatening lower respiratory tract infections can sometimes occur in infants, elderly people, or immunocompromised patients (Encyclopedia of Virology. 2021:428-440).

As used herein, the terms “severe acute respiratory syndrome coronavirus 2 (SARS-CoV-2)” refers to the strain of coronavirus that causes coronavirus disease 2019 (COVID-19). SARS-CoV-2 is a positive-sense single-stranded RNA virus, with a genome size of 29,903 bases. Each SARS-CoV-2 virion is 50-200 nanometres in diameter, with four structural proteins, known as the S (spike), E (envelope), M (membrane), and N (nucleocapsid) proteins. The N protein holds the RNA genome, and the S, E, and M proteins together create the viral envelope. The spike protein is the protein responsible for allowing the virus to attach to and fuse with the membrane of a host cell; specifically, its S1 subunit catalyzes attachment, the S2 subunit fusion.

As used herein, the term “major histocompatibility complex (MHC)” refers to a group of genes that code for proteins found on the surfaces of cells that help the immune system recognize foreign substances. MHC proteins are found in all higher vertebrates. In human beings the complex is also called the human leukocyte antigen (HLA) system. MHC is mainly present in a form binding to a membrane and is responsible for the regulation of immune system. MHC activates cytotoxic T-cells by presenting a peptide fragment of foreign or autologous protein on the cell surface and allowing the T Cell Receptor (TCR) of the cytotoxic T-cell to recognize and bind it. MHC may be mainly divided into two types. MHC class I is present on the surface of almost all cells, whereas MHC class II presents only in an antigen presenting cell (APC) such as NK cells, macrophages, and dendritic cells. The MHC class I can selectively activate cytotoxic T-cells expressing TCR when a specific peptide fragment is loaded thereto, and thus the recombinant MHC class I in which the membrane-bound domain has been removed is used for the treatment of cancer or infectious disease. Unlike MHC class II, most of the MHC class I is unstable when the peptide is not loaded in the groove. Therefore, recombinant MHC class I is prepared in a form loading the target synthetic peptide or in a form fused to the a chain. In addition, since there is a risk that refolding may not be properly performed when expressed using a microorganism, it is generally expressed using animal cells. In the art, HLA and MHC are used interchangeably with the same meaning, and HLA is used instead of MHC in human. In the present specification, the terms “HLA” and “MHC” may be used interchangeably with the same meaning.

As used herein, the nomenclature used to describe peptides of the invention follows the conventional practice wherein the amino group (N-terminus) and/or the 5′ are presented to the left and the carboxyl group (C-terminus) and/or 3′ are presented to the right.

As used herein, the term “peptide” refers to a molecular chain of amino acids, including both L-forms and D-forms. The amino acids, if required, can be modified in vivo or in vitro, for example by manosylation, glycosylation, amidation (specifically C-terminal amides), carboxylation or phosphorylation with the stipulation that these modifications must preserve the biological activity of the original molecule. In addition, peptides can be part of a chimeric protein.

Functional derivatives of the peptides are also included in the present invention. Functional derivatives are meant to include peptides which differ in one or more amino acids in the overall sequence, which have deletions, substitutions, inversions or additions. Amino acid substitutions which can be expected not to essentially alter biological and immunological activities have been described. Amino acid replacements between related amino acids or replacements which have occurred frequently in evolution include, inter alia Ser/Ala, Ser/Gly, Asp/Gly, Asp/Asn and Ile/Val.

The peptides according to the invention can be produced synthetically or by recombinant DNA technology. Methods for producing synthetic peptides are well known in the art.

The organic chemical methods for peptide synthesis are considered to include the coupling of the required amino acids by means of a condensation reaction, either in homogenous phase or with the aid of a so-called solid phase. The condensation reaction can be carried out as follows: Condensation of a compound (amino acid, peptide) with a free carboxyl group and protected other reactive groups with a compound (amino acid, peptide) with a free amino group and protected other reactive groups, in the presence of a condensation agent. Condensation of a compound (amino acid, peptide) with an activated carboxyl group and free or protected other reaction groups with a compound (amino acid, peptide) with a free amino group and free or protected other reactive groups. Activation of the carboxyl group can take place, inter alia, by converting the carboxyl group to an acid halide, azide, anhydride, imidazolide or an activated ester, such as the N-hydroxy-succinimide, N-hydroxy-benzotriazole or p-nitrophenyl ester.

The most common methods for the above condensation reactions are: the carbodiimide method, the azide method, the mixed anhydride method and the method using activated esters, such as described in The Peptides, Analysis, Synthesis, Biology Vol. 1-3 (Ed. Gross, E. and Meienhofer, J.) 1979, 1980, 1981 (Academic Press, Inc.).

By a “variant” of the given amino acid sequence the inventors mean that the side chains of, for example, one or two of the amino acid residues are altered (for example by replacing them with the side chain of another naturally occurring amino acid residue or some other side chain) such that the peptide is still able to bind to an MHC molecule in substantially the same way as a peptide consisting of the given amino acid sequence in consisting of SEQ ID NO: 1 to SEQ ID NO: 26. For example, a peptide may be modified so that it at least maintains, if not improves, the ability to interact with and bind to the binding groove of a suitable MHC molecule, such as HLA-A*02.

A person skilled in the art will be able to assess, whether T cells induced by a variant of a specific peptide will be able to cross-react with the peptide itself.

The present invention also relates to a nucleic acid encoding the peptide of the present invention. Therefore, the nucleic acid encodes a peptide comprising or consisting of any one of SEQ ID NOs: 1 to 26, or a variant thereof.

As used herein, the term “nucleic acid encoding a peptide” refers to a nucleotide sequence encoding for the peptide. The nucleic acid encoding a particular peptide, oligopeptide, or polypeptide may be naturally occurring or they may be synthetically constructed. The nucleic acid (for example a polynucleotide) may be, for example, deoxyribonucleic acid (DNA), complementary DNA (cDNA), peptide nucleic acid (PNA), ribonucleic acid (RNA), or combinations thereof, either single- and/or double-stranded, or native or stabilized forms of polynucleotides, such as, for example, polynucleotides with a phosphorothioate backbone and it may or may not contain introns so long as it codes for the peptide.

The present invention also relates to an immunogenic composition, comprising the peptide of the present invention or the nucleic acid of the present invention. Therefore, the immunogenic composition comprises a peptide comprising or consisting of any one of SEQ ID NOs: 1 to 26, or a variant thereof. Alternatively, the immunogenic composition comprises nucleic acid encodes a peptide comprising or consisting of any one of SEQ ID NOs: 1 to 26, or a variant thereof.

In some embodiments, the immunogenic composition further comprises a pharmaceutically acceptable carrier and/or an adjuvant.

In some embodiments, the immunogenic composition comprises at least one peptide of the present invention. In some embodiments, the immunogenic composition comprises two or more peptides of the present invention. The immunogenic composition comprising the peptide of the present invention is also called a peptide vaccine.

As used herein, the term “peptide vaccine” refers to a preparation composed of at least one peptide that improves immunity to a particular pathogen.

In some embodiments, the immunogenic composition comprises nucleic acid encoding at least one peptide of the present invention. In some embodiments, the immunogenic composition comprises nucleic acid encoding two or more peptides of the present invention. In some embodiments, the nucleic acid is DNA. In some embodiments, the nucleic acid is RNA. In some embodiments, the nucleic acid is mRNA.

The term “immunogenic composition” as used herein refers to a composition that is able to produce an immune response.

In some embodiments, the immunogenic composition exhibits, upon administration, activation of T cells. In some embodiments, the immunogenic composition exhibits, upon administration, activation of CD4⁺ T cells. In some embodiments, the immunogenic composition exhibits, upon administration, activation of CD8⁺ T cells. In some embodiments, the immunogenic composition exhibits, upon administration, combined activation of CD4⁺ and CD8⁺ T cells.

In some embodiments, the immunogenic composition exhibits, upon administration, production of specific antibodies (of any immunoglobin class) against epitopes within the peptides of the present invention. Each possibility represents a separate embodiment.

The term “adjuvant” as used herein refers to any component of a pharmaceutical composition that is not the active agent.

In some embodiments, the adjuvant is selected from the group comprising an oil emulsion, a cytokine, an immunostimulating complex (ISCOM), a saponin-type auxiliary agent, Montanide ISA 51VG, liposomes, aluminum hydroxide (alum), bovine serum albumin (BSA), keyhole limpet hemocyanin (KLH), Lipopolysacharrudes (LPS) or derivatives such as Monophosphoryl lipid A (MPL), CpG DNA, microbial DNA/RNA, nanoparticle (e.g., gold particles), bacterial ghosts, ligands or agonist antibodies for TNFa, TLR (Toll-like receptor-based adjuvants (e.g. see Heit at al., Eur. J. Immunol., 2007, 37:2063-2074) or a combination thereof.

As used herein, “pharmaceutically acceptable carrier” or “pharmaceutically acceptable excipient” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal or transdermal administration. Depending on the route of administration, the active compound may be coated in a material to protect the compound from the action of acids and other natural conditions which may inactivate the compound. The use of such media and agents for pharmaceutically active substances is well known in the art.

In some embodiments, peptide antigens are associated with polymer, such as Poly(lactide-co-caprolactone)-block-poly(ethylene glycol)-block-poly(lactide-co-caprolactone) (PLCL-PEG-PLCL), Poly(ε-caprolactone)-poly(ethylene glycol)-poly(ε-caprolactone) (PCL-PEG-PCL) or other polymers known in the art, for peptide encapsulation and delivery, such as, but not limited to, carboxymethylcellulose (CMC), chitosan, and 1,2-distearoyl-sn-glycero-3-phosphocholine (DSPC).

In some embodiments, no adjuvant is added. In some embodiments, one adjuvant is added. In some embodiments, a combination of adjuvants is added.

The present invention also relates to an antibody specifically recognizing the peptide of the present invention. Therefore, the antibody specifically recognizes a peptide comprising or consisting of any one of SEQ ID NOs: 1 to 26, or a variant thereof.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2, single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “antibody” or “antibodies” is used herein in a broad sense and includes both polyclonal and monoclonal antibodies. In addition to intact or “full” immunoglobulin molecules, also included in the term “antibodies” are fragments (e.g. CDRs, Fv, Fab and Fc fragments) or polymers of those immunoglobulin molecules and humanized versions of immunoglobulin molecules, as long as they exhibit any of the desired properties, i.e. specifically recognize the peptide or variant thereof according to the invention. Whenever possible, the antibodies of the invention may be purchased from commercial sources. The antibodies of the invention may also be generated using well-known methods. The antibodies of the present invention can be used as therapeutics or diagnostics.

The present invention also relates to a T-cell receptor being capable of binding to the peptide of the present invention. Therefore, the T-cell receptor is capable of binding to a peptide comprising or consisting of any one of SEQ ID NOs: 1 to 26, or a variant thereof.

In some embodiments, the peptide is bound to an MHC molecule.

As used herein, the term “T-cell receptor” (TCR) refers to a heterodimeric molecule comprising an alpha polypeptide chain (alpha chain) and a beta polypeptide chain (beta chain), wherein the heterodimeric receptor is capable of binding to a peptide antigen presented by an HLA molecule. The term also includes so-called gamma/delta TCRs.

The present invention also relates to a recombinant host cell, comprising one component selected from the group consisting of the peptide of the present invention, the nucleic acid of the present invention, the antibody or fragment thereof of the present invention, and the T-cell receptor or fragment thereof of the present invention. Therefore, the recombinant host cell comprises one component selected from the group consisting of a peptide comprising or consisting of any one of SEQ ID NOs: 1 to 26, or a variant thereof, nucleic acid encoding a peptide comprising or consisting of any one of SEQ ID NOs: 1 to 26, or a variant thereof, an antibody or fragment specifically recognizes a peptide comprising or consisting of any one of SEQ ID NOs: 1 to 26, or a variant thereof, and a T-cell receptor being capable of binding to a peptide comprising or consisting of any one of SEQ ID NOs: 1 to 26, or a variant thereof.

In some embodiments, the recombinant host cell is selected from an antigen presenting cell, such as a dendritic cell, a T cell, or a natural killer (NK) cell.

The present invention also relates to an in vitro or ex vivo method for producing activated T lymphocytes, comprising contacting in vitro or ex vivo T cells with antigen loaded human class I or II MHC molecules expressed on the surface of a suitable antigen-presenting cell or an artificial construct mimicking an antigen-presenting cell for a period of time sufficient to activate the T cells in an antigen specific manner, wherein the antigen is the peptide of the present invention.

The present invention also relates to an activated T lymphocyte produced by the in vitro or ex vivo method mentioned above, wherein the activated T lymphocyte selectively recognizes a cell presenting the peptide of the present invention. Therefore, the activated T lymphocyte selectively recognized a cell presenting a peptide comprising or consisting of any one of SEQ ID NOs: 1 to 26, or a variant thereof.

The present invention also relates to a pharmaceutical composition, comprising at least one active ingredient selected from the group consisting of the peptide of the present invention, the nucleic acid of the present invention, the antibody or fragment thereof of the present invention, the T-cell receptor or fragment thereof of the present invention, the recombinant host cell of the present invention, and the activated T lymphocyte of the present invention. Therefore, the pharmaceutical composition comprises at least one active ingredient selected from the group consisting of a peptide comprising or consisting of any one of SEQ

ID NOs: 1 to 26, or a variant thereof, nucleic acid encoding a peptide comprising or consisting of any one of SEQ ID NOs: 1 to 26, or a variant thereof, an antibody or fragment specifically recognizes a peptide comprising or consisting of any one of SEQ ID NOs: 1 to 26, or a variant thereof, and a T-cell receptor being capable of binding to a peptide comprising or consisting of any one of SEQ ID NOs: 1 to 26, or a variant thereof.

In some embodiments, the pharmaceutical composition further comprises a pharmaceutically acceptable carrier, and/or pharmaceutically acceptable excipients and/or stabilizers.

As used herein, the term “pharmaceutical composition” refers to a composition suitable for administration to a human being in a medical setting. Preferably, a pharmaceutical composition is sterile and produced according to GMP guidelines.

In preparing pharmaceutical compositions of the present invention, it may be desirable to modify the peptide antigen, or to combine or conjugate the peptide with other agents, to alter pharmacokinetics and biodistribution. A number of methods for altering pharmacokinetics and biodistribution are known to persons of ordinary skill in the art. Examples of such methods include protection of the proteins, protein complexes and polynucleotides in vesicles composed of other proteins, lipids (for example, liposomes), carbohydrates, or synthetic polymers. For example, the vaccine agents of the invention can be incorporated into liposomes in order to enhance pharmacokinetics and biodistribution characteristics. A variety of methods are available for preparing liposomes, as described in, e.g., U.S. Pat. Nos. 4,235,871, 4,501,728 and 4,837,028. For use with liposome delivery vehicles, peptides are typically entrapped within the liposome, or lipid vesicle, or are bound to the outside of the vesicle.

The present invention also relates to a method for preventing or treating a pathogenic infection in a subject in need thereof, comprising administering to the subject an effective amount of the immunogenic composition or the pharmaceutical composition of the present invention.

In some embodiments, the pathogenic infection is induced by a coronavirus. In some embodiments, the coronavirus is the severe acute respiratory syndrome coronavirus 2 (SARS-CoV-2).

In some embodiments, the present invention provides a method of treating or preventing a pathogenic infection comprising administering an enriched T cell population to a subject in need thereof, wherein the enriched T cell population is obtained by administering the immunogenic composition to a T cell population in vitro.

As used herein, an “effective amount” or a “sufficient amount” of a substance is that amount sufficient to effect beneficial or desired results, including clinical results, and, as such, an “effective amount” depends upon the context in which it is being applied. In the context of administering an immunogenic composition, the effective amount is an immunogenically effective amount, which contains sufficient immunogenic composition of the present invention to elicit an immune response. In the context of administering a pharmaceutical composition, the effective amount is a pharmaceutically effective amount, which contains sufficient pharmaceutical composition of the present invention to maintain or produce a desired physiological result. An effective amount can be administered in one or more doses.

As used herein, the term “immunogenically effective amount” refers to an amount that is, in combination, effective, at dosages and for periods of time necessary, to elicit a specific T lymphocyte mediated immune response and/or a humoral response. This response can be determined by conventional assays for T-cell activation, including but not limited to assays to detect antibody production, proliferation, specific cytokine activation and/or cytolytic activity, e.g., using an antibody concentration/titer assay (e.g. via ELISA).

As used herein, the term “pharmaceutically effective amount” refers to an amount capable of or sufficient to maintain or produce a desired physiological result, including but not limited to treating, reducing, eliminating, substantially preventing, or prophylaxing, or a combination thereof, a disease, disorder, or combination thereof. A pharmaceutically effective amount may comprise one or more doses administered sequentially or simultaneously. Those skilled in the art will know to adjust doses of the present invention to account for various types of formulations, including but not limited to slow-release formulation. As used herein, the term “prophylactic” refers to a composition capable of substantially preventing or prophylaxing any aspect of a disease, disorder, or combination thereof. As used herein, the term “therapeutic” refers to a composition capable of treating, reducing, halting the progression of, slowing the progression of, beneficially altering, eliminating, or a combination thereof, any aspect of a disease, disorder, or combination thereof.

The term “dose” as used herein in reference to an immunogenic composition refers to a measured portion of the immunogenic composition taken by (administered to or received by) a subject at any one time.

As used herein the term “immunization” refers to a process that increases a mammalian subject's reaction to antigen and therefore improves its ability to resist or overcome infection.

The term “vaccination” as used herein refers to the introduction of vaccine into a body of a mammalian subject.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In some embodiments, the subject is a human. Human subjects may also include fetuses.

As used herein, the terms “subject,” refers to any subject, particularly a mammalian subject, for whom therapy is desired, for example, a human.

In some embodiments, a subject in need thereof is afflicted with a pathogenic infection. In some embodiments, a subject in need thereof is susceptible to a pathogenic infection. In some embodiments, a subject in need thereof is potentially susceptible to a pathogenic infection.

The term “treat,” “treating,” or “treatment” as used herein encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

In some embodiments, the peptide vaccine of the invention reduces transfection or transmission to other subjects.

In some embodiments, the peptide vaccine of the invention is administered in an effective amount with or without a co-stimulatory molecule, agent or adjuvant. According to the method of the invention, the peptide vaccine may be administrated to a subject in need of such treatment for a time and under conditions sufficient to prevent, and/or ameliorate the pathogen infection.

In some embodiments, the immunogenic composition may be administered to subjects by a variety of administration modes, including by intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes. Alternatively, the antigen may be administered ex-vivo by direct exposure to cells, tissues or organs originating from a subject (autologous) or another subject (allogeneic), optionally in a biologically suitable, liquid or solid carrier.

Peptide vaccines may be administered to the subject per se or in combination with an appropriate auxiliary agent or adjuvant via injection. Alternatively, the peptide vaccine may be percutaneously administered through mucous membrane by, for instance, spraying the solution. The unit dose of the peptide typically ranges from about 0.001 mg to 100 mg, more typically between about 1 μg to about 1,000 μg, which may be administered, one time or repeatedly, to a patient.

Examples of auxiliary agents or adjuvants which can be formulated with or conjugated to peptide or protein antigens and/or vectors for expressing co-stimulatory molecules to enhance their immunogenicity for use within the invention include cytokines (e.g. GM-CSF), bacterial cell components such as BCG bacterial cell components, immnunostimulating complex (ISCOM), extracted from the tree bark called QuillA, QS-21, a saponin-type auxiliary agent, Montanide ISA 51VG, liposomes, aluminum hydroxide (alum), bovine serum albumin (BSA), tetanus toxoid (TT), keyhole limpet hemocyanin (KLH), and TLR (Toll-like receptor)-based adjuvants (e.g. see Heit at al Eur. J. Immunol. (2007) 37:2063-2074).

The present invention also relates to a method for generating anti-coronavirus antibodies, comprising administering the immunogenic composition of the present invention to an animal or a human subject.

In some embodiments, the anti-coronavirus antibodies are characterized by having binding affinity to a coronavirus. In some embodiments, the anti-coronavirus antibodies are antibodies against alpha coronavirus peptides. In some embodiments, the coronavirus is 229E or NL63. In some embodiments, the anti-coronavirus antibodies are antibodies against beta coronavirus peptides. In some embodiments, the coronavirus is OC43, HKU1, MERS-CoV, SARS-CoV and SARS-CoV-2.

In some embodiments, the animal is a horse, cow, camel, goat, sheep, dog, cat, non-human primate, mouse, rat, rabbit, hamster, guinea pig or pig. An animal may also include prenatal forms of animals, such as, e.g., embryos or fetuses.

The present invention also relates to a complex comprising the peptide of the present invention bound to an MHC molecule. Therefore, the complex comprises a peptide comprising or consisting of any one of SEQ ID NOs: 1 to 26, or a variant thereof, bound to an MHC molecule.

In some embodiments, the MHC molecule is an MHC class 1 molecule. In some embodiments, the MHC molecule is an MHC class II molecule. Preferably, the MHC molecule is an MHC class I molecule. The MHC class I molecule may be of any HLA supertype. For example, the MHC class I molecule may be of supertype A2.

In some embodiments, the complex comprises two or more peptides of the present invention and two or more MHC molecules. For example, the complex may comprise three or more, such as four or more, five or more, six or more, seven or more, eight or more, nine or more, ten or more peptides of the invention. The complex may comprise three or more, such as four or more, five or more, six or more, seven or more, eight or more, nine or more, ten or more MHC molecules. The complex may, for example, comprise three or more, such as four or more, five or more, six or more, seven or more, eight or more, nine or more, ten or more peptides of the invention and three or more, such as four or more, five or more, six or more, seven or more, eight or more, nine or more, ten or more MHC molecules, respectively. The complex may comprise the same number of peptides of the invention as MHC molecules. The complex may comprise a different number of peptides of the invention from the number MHC molecules. The complex may, for example, comprise four MHC molecules. The complex may comprise or consist of an MHC tetramer. The complex may, for example, comprise twelve MHC molecules. The complex may comprise or consist of an MHC dodecamer.

When the complex comprises two or more peptides of the invention, each of the two or more peptides may be the same. Alternatively, each of the two or more peptides may be different. When the complex comprises three or more peptides of the invention, each of the three or more peptides may be the same. When the complex comprises three or more peptides of the invention, each of the three or more peptides may be different. When the complex comprises three or more peptides of the invention, some of the three or more peptides may be the same and some of the three of more peptides may be different.

When the complex comprises two or more MHC molecules, each of the two or more MHC molecules may be the same. Alternatively, each of the two or more MHC molecules may be different. When the complex comprises three or more peptides of the invention, each of the three or more MHC molecules may be the same. When the complex comprises three or more peptides of the invention, each of the three or more MHC molecules may be different. When the complex comprises three or more MHC molecules, some of the three or more MHC molecules may be the same and some of the three of more MHC molecules may be different.

In some embodiments, the complex comprises two or more peptides of the invention and two or more MHC molecules, and each peptide may be bound to one of the two or more MHC molecules. That is, each peptide comprised in the complex may be bound to an MHC molecule comprised in the complex. Preferably, each peptide comprised in the complex is bound to a different MHC molecule comprised in the complex. That is, each MHC molecule comprised in the complex is preferably bound to no more than one peptide comprised in the complex. The complex may, however, comprise one or more peptides of the invention that are not bound to an MHC molecule. The complex may comprise one or more MHC molecules that are not bound to a peptide of the invention.

The MHC molecule or molecules comprised in the complex may be linked to one another. For example, each of the one or more MHC molecules in the complex may be attached to a backbone molecule or a nanoparticle. In some embodiments, each of the two or more MHC molecules is attached to a dextran backbone. That is, the complex may comprise or consist of an MHC dextramer. Mechanisms for attaching an MHC molecule or molecules to a dextran backbone are known in the art. Any number of MHC molecules may be attached to the dextran backbone. For example, one or more, two or more, three or more peptides of the invention and three or more MHC molecules may be attached to the dextran backbone.

In some embodiments, the complex further comprises a fluorophore, optionally wherein the fluorophore is attached to the dextran backbone. Fluorophores are well-known in the art and include FITC (fluorescein isothiocyanate), PE (phycoerythrin) and APC (allophycocyanin). The complex may comprise any number of fluorophores. For example, the complex may comprise two or more, three or more, such as four or more, five or more, six or more, seven or more, eight or more, nine or more, ten or more peptides of the invention and three or more, such as four or more, five or more, six or more, seven or more, eight or more, nine or more, ten or more fluorophores. When the complex comprises multiple fluorophores, the fluorophores comprised in the complex may be the same or different. When the complex comprises a backbone, such as a dextran backbone, the fluorophore is preferably attached to the dextran backbone. Mechanisms for attaching a fluorophore to a dextran backbone are known in the art.

The present invention also relates to a method for determining the presence or absence of current or previous coronavirus infection in an individual, comprising contacting the peptide or the complex of the present invention with a sample obtained from the individual and determining the presence or absence of binding between the peptide or complex and a molecule comprised in the sample.

The sample may, for example, be a blood sample, a serum sample, a plasma sample, a urine sample, a saliva sample, or a sample obtained by swabbing a mucosal surface present in the individual. Preferably, the sample is a blood sample, a serum sample, or a plasma sample.

In some embodiments, the molecule is an antibody or a T cell receptor. The molecule may, for example, be an antibody or antibody fragment. The antibody or antibody fragment may be on the surface of, or comprised in, a B cell. The antibody or antibody fragment may be free in the sample. The molecule may, for example, be a T cell receptor. The T cell receptor may be a CD4⁺ T cell receptor. The T cell receptor may be a CD8⁺ T cell receptor. The T cell receptor may be on the surface of, or comprised in, a T cell. The T cell may be a CD4⁺ T cell. The T cell may be a CD8⁺ T cell.

Methods for detecting binding between a peptide or peptide-containing complex and a molecule are well-known in the art in include, for example, enzyme-linked immunosorbent assay (ELISA), enzyme-linked immune absorbent spot (ELISpot), and flow cytometry.

In some embodiments, the presence of binding indicates the presence of current or previous coronavirus infection, and/or the absence of binding indicates the absence of current or previous coronavirus infection.

In a current coronavirus infection, coronavirus particles or components thereof (e.g. peptides, proteins) may be present within the individual. In a current coronavirus infection, antibodies, B cells, CD8⁺ T cells and/or CD4⁺ T cells specific for coronavirus particles or components thereof (e.g. peptides, proteins) may be present within the individual. Preferably, in a current coronavirus infection (i) coronavirus particles or components thereof (e.g. peptides, proteins) and (ii) antibodies, B cells, CD8⁺ T cells and/or CD4⁺ T cells specific for coronavirus particles or components (e.g. proteins) thereof are present within the individual.

In a previous coronavirus infection, coronavirus particles or thereof components (e.g. peptides, proteins) may be absent from the individual. In a previous coronavirus infection, antibodies, B cells, CD8⁺ T cells and/or CD4⁺ T cells specific for coronavirus particles or components thereof (e.g. peptides, proteins) may be present within the individual. Preferably, in a previous coronavirus infection, coronavirus particles or components thereof (e.g. peptides, proteins) are absent from the individual, and antibodies, B cells, CD8⁺ T cells and/or CD4⁺ T cells specific for coronavirus particles or components thereof (e.g. peptides, proteins) are present within the individual.

The present invention also relates to a method for identifying coronavirus-specific T cells, comprising contacting the peptide or the complex of the present invention with a sample obtained from an individual and determining the presence or absence of binding between the peptide or complex and a T cell receptor comprised in the sample.

The sample may, for example, be a blood sample, a serum sample, a plasma sample, a urine sample, a saliva sample, or a sample obtained by swabbing a mucosal surface present in the individual. Preferably, the sample is a blood sample.

The T cell receptor may be a CD4⁺ T cell receptor. The T cell receptor may be a CD8⁺ T cell receptor. Preferably, the T cell receptor is a CD8⁺ T cell receptor.

The T cell receptor may be on the surface of, or comprised in, a T cell. The T cell may be a CD4⁺ T cell. The T cell may be a CD8⁺ T cell. Preferably, the T cell is a CD8⁺ T cell.

Methods for detecting binding between a peptide or peptide-containing complex and a T cell receptor are well-known in the art in include, for example, enzyme-linked immunosorbent assay (ELISA), enzyme-linked immune absorbent spot (ELISpot), and flow cytometry.

The presence of binding may indicate the presence of one or more coronavirus-specific T cells. The absence of binding may indicate the absence of coronavirus-specific T cells.

In some embodiments, the individual is currently infected with the coronavirus. In a current coronavirus infection, coronavirus particles or components thereof (e.g. peptides, proteins) may be present within the individual. In a current coronavirus infection, antibodies, B cells, CD8⁺ T cells and/or CD4⁺ T cells specific for coronavirus particles or components thereof (e.g. peptides, proteins) may be present within the individual. Preferably, in a current coronavirus infection (i) coronavirus particles or components thereof (e.g. peptides, proteins) and (ii) antibodies, B cells, CD8⁺ T cells and/or CD4⁺ T cells specific for coronavirus particles or components thereof (e.g. peptides, proteins) are present within the individual.

In some embodiments, the individual was previously, but is not currently, infected with the coronavirus. In a previous coronavirus infection, coronavirus particles or components thereof (e.g. peptides, proteins) may be absent from the individual. In a previous coronavirus infection, antibodies, B cells, CD8⁺ T cells and/or CD4⁺ T cells specific for coronavirus particles or components thereof (e.g. peptides, proteins) may be present within the individual. Preferably, in a previous coronavirus infection, coronavirus particles or components thereof (e.g. peptides, proteins) are absent from the individual, and antibodies, B cells, CD8⁺ T cells and/or CD4⁺ T cells specific for coronavirus particles or components thereof (e.g. peptides, proteins) are present within the individual. Thus, coronavirus particles or components thereof (e.g. peptides, proteins) may be absent from the individual but antibodies, B cells, CD8⁺ T cells and/or CD4⁺ T cells specific for coronavirus particles or components thereof (e.g. peptides, proteins) may be present within the individual.

The present invention also relates to a method for identifying a coronavirus-specific T cell receptor, comprising contacting the peptide or the complex of the present invention with a T cell receptor and determining the presence or absence of binding between the peptide or complex and the T cell receptor.

In some embodiments, the presence of binding indicates that the T cell receptor is a coronavirus-specific T cell receptor, and/or the absence of binding indicates that the T cell receptor is not coronavirus-specific T cell receptor.

The T cell receptor may be a CD4⁺ T cell receptor. The T cell receptor may be a CD8⁺ T cell receptor. Preferably, the T cell receptor is a CD8⁺ T cell receptor.

The T cell receptor may be on the surface of, or comprised in, a T cell. The T cell may be a CD4⁺ T cell. The T cell may be a CD8⁺ T cell. Preferably, the T cell is a CD8⁺ T cell.

Methods for detecting binding between a peptide or peptide-containing complex and a T cell receptor are well-known in the art in include, for example, enzyme-linked immunosorbent assay (ELISA), enzyme-linked immune absorbent spot (ELISpot), and flow cytometry.

The meaning of the technical and scientific terms as described herein can be clearly understood by a person of ordinary skill in the art.

As used herein, the term “about,” “around,” or “approximately” when combined with a value refers to plus and minus 10% of the reference value. For example, a length of about 1000 nanometers (nm) refers to a length of 1000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1 Binding Ability Assay of the Antigen Peptides to MHC Class I

Materials and Methods

MHC-peptide binding assay. An MHC-peptide binding assay was used to determine the ability of each antigen peptide of the present invention to bind HLA-A201, which is the most common human MHC class I allele in the population (Wills, M. R. et al, J. Virol. 1996, 70:7569-7579; Weekes, M. P. et al., J. Virol. 1999, 73:2099-2108; Reiser, J. B. et al., Acta Cryst. Sect. F Struct. Biol. Cryst. Commun. 2009, 65:1157-1161). MHC class I refolding assay (easYmer, immunAware Aps, Copenhagen, Denmark) was preformed according to manufacturer's instruction. Briefly, each antigen peptide of the present invention (SEQ ID NOs: 1-26) was incubated with β2-microglobulin light chain subunit and biotin-labeled recombinant HLA-A201 at 18° C. for 48 hours to form a peptide-HLA complex. The biotin-labeled peptide-HLA complex was then captured by streptavidin-coated beads and detected by PE-conjugated anti-human β2-microglobulin on flow cytometry. A cytomegalovirus (CMV) pp65 protein-derived peptide (⁴⁹⁵NLVPMVATV⁵⁰³; SEQ ID NO: 27; hereafter, CMVpp65₄₉₅₋₅₀₃), which is a known HLA-A201 strong binding peptide having a binding affinity (IC50) of 45 nM for HLA-A201 (Hassan C. et al., J. Biol. Chem. 2015, 290:2593-2603), was used as the positive control. No peptide was used in the negative control.

Results

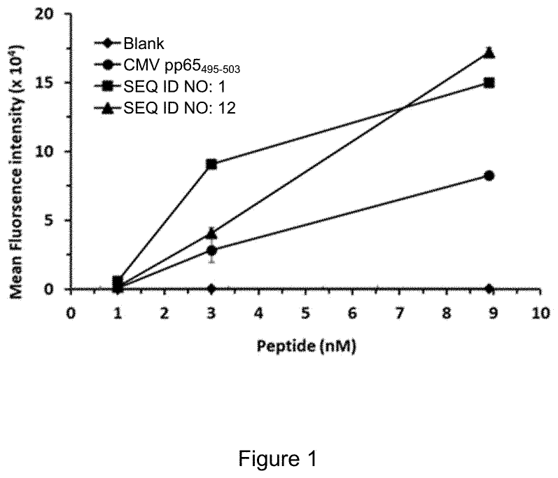

The peptides of the present invention have binding ability to human MHC class I As shown in FIG. 1, both Peptide 1 (SEQ ID NO: 1) and Peptide 12 (SEQ ID NO: 12) of the present invention have higher binding affinity for HLA-A201 than CMVpp65₄₉₅₋₅₀₃ (SEQ ID NO: 27; positive control) at the concentration of 3 nM and 8.9 nM. In particular, the binding affinity of 8.9 nM Peptide 1 (SEQ ID NO: 1) and Peptide 12 (SEQ ID NO: 12) for HLA-A201 is 1.8 and 2.1 times as high as that of CMVpp65₄₉₅₋₅₀₃ (SEQ ID NO: 27; positive control), respectively.

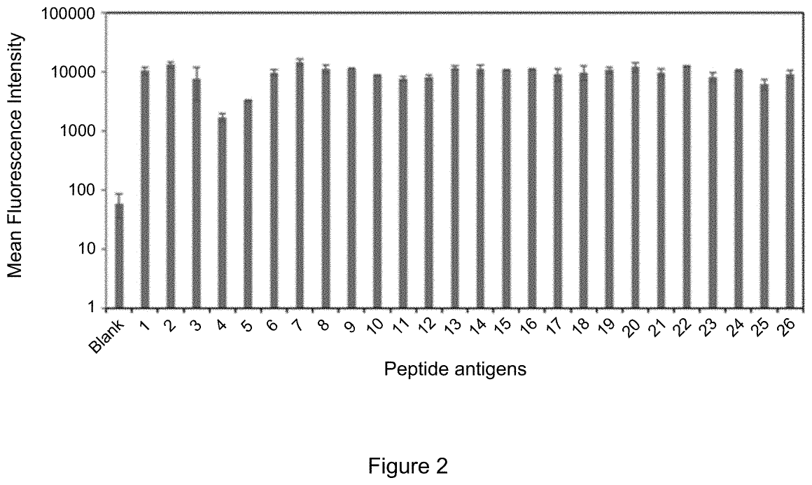

In addition, as shown in FIG. 2, all of the antigen peptides of the present invention (SEQ ID NOs: 1-26) show strong binding affinity for HLA-A201 at the concentration of 8.9 nM.

The results of Example 1 indicate that the peptides of the present invention (SEQ ID NOs: 1-26) have strong binding affinity for HLA-A201, which is the most common human MHC class I allele in the population. Therefore, when one of the peptides of the present invention is administrated into a population of human subjects, it binds the MHC class I in most of the subjects to trigger the subsequent immune responses.

### Example 2 MHC Class I Tetramer Assay

MHC tetramer reagents allow rapid and simple detection of antigen-specific T cells. MHC tetramer technology is based on the ability of MHC-peptide complexes to recognize the antigen-specific T cells at a single cell level. This technology enables researchers to precisely measure involving responsible T-cell responses in infectious diseases, cancer, and autoimmune diseases. The existence of antigen-specific T-cell immune responses is thought to be the most important and relevant outcome of anti-tumor or anti-viral responses for the development of vaccines and therapies. Due to the importance and relevance of the existence of antigen-specific T-cell immune response for the development of vaccines, an MHC tetramer assay was used to detect antigen-specific T-cells in this Example.

Materials and Methods

MHC class I tetramer assay. MHC class I tetramer assay (easYmer®, immunAware Aps, Copenhagen, Denmark) was preformed according to manufacturer's instruction. Briefly, 3 μL of 75 μM Peptide 1 (SEQ ID NO: 1) or Peptide 12 (SEQ ID NO: 12) of the present invention were mixed with 45 μL of ddH2O, 12 μL of Folding Buffer (X6), and 12 μL of easYmer® and incubated at 18° C. for 48 hours to obtain around 500 nM folded monomer. Then, 504 of the obtained folded monomer were mixed with the equivalent of 2.1 μL of a 0.2 mg/mL streptavidin-fluorophore and incubated in the dark at 4° C. for at least 1 hour to tetramerized the monomer. The tetramer was diluted to 30 nM in FACS buffer (PBS with 1% v/v BSA and 0.1% v/v NaN₃) for staining of human T cells.

Next, around 1-2×10⁵ human peripheral blood mononuclear cells (PBMCs, HLA-A201⁺) (STEMCELL Technologies Inc, Vancouver, British Columbia, Canada) in FACS buffer were seeded onto a 96-well microplate. The cells were centrifuged at 700×g for 3 minutes, and the supernatant was removed. The cells were resuspended with 40 μL of the diluted tetramer and incubated in the dark at room temperature (RT) for 20 minutes. The cells were washed once in cold FACS buffer and centrifuged at 700×g for 3 minutes. After the supernatant was removed, the cells were co-stained with anti-CD8 antibodies and incubated in the dark at 4° C. for 30 minutes. The cells were washed twice in cold FACS buffer, resuspended in FACS buffer, and analyzed in a flow cytometer (BD LSRFortessa™X20, Franklin Lakes, N.J., U.S.).

Statistical analysis. Microsoft Excel was used for statistical analysis. Student t-test was used to calculate significance. * p<0.05, ** p<0.01, *** p<0.001.

Results

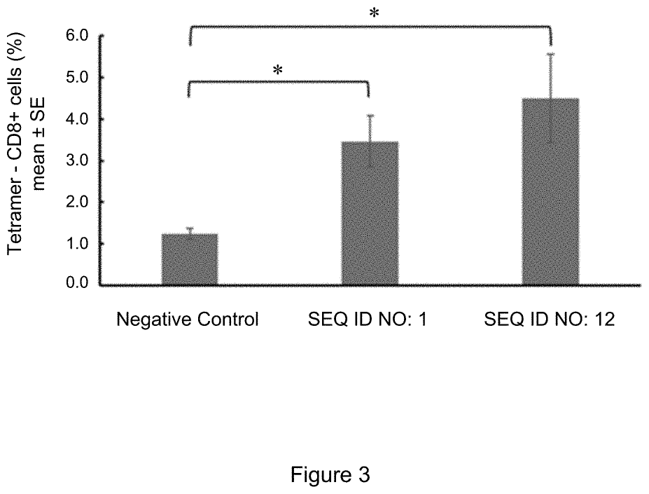

The peptides of the present invention induce antigen-specific T-cell immune response. As shown in FIG. 3, antigen specific cytotoxic T cells (CTLs) of Peptide 1 (SEQ ID NO: 1) and Peptide 12 (SEQ ID NO: 12) of the present invention can be detected by MHC class I tetramer staining in human PBMC (HLA-A201⁺). The results indicate that the antigen peptides of the present invention induce at least antigen specific CTLs (i.e., CD8⁺ T cell) immune response. The peptide-tetramer complex can detect the corresponding specific CTLs, and these responding CTLs can recognize exogenous organism or endogenous cells bearing the peptides. Through the recognition of CTLs to the peptides, the organism or infected cells can be eliminated.

In addition, the results show that the HLA class I tetramer binding to Peptide 1 (SEQ ID NO: 1) or Peptide 12 (SEQ ID NO: 12) engages around 2.2 to 3.3% CD8⁺ T cells in the human PBMCs. Since it is estimated that around 4×10¹⁰ CD8⁺ T cells present in an adult human (Alanio, C. et al., Blood, 2010, 115(18):3718-3725), the peptides of the present invention are able to engage around 0.9 to 1.3×10⁹ CD8⁺ T cells in an adult human body in recognizing virus-infected cells and inducing apoptosis in the cells.

### Example 3 Intracellular Cytokine Assay

To further verify the ability of the antigen peptides to induce T cell response, intracellular cytokine staining was used to detect the cytokine production of CD4⁺ T helper cells in response to the peptides of the present invention.

Materials and Methods

Intracellular cytokine staining and multiparameter flow cytometry. Around 1-2×10⁵ human PBMCs (HLA-A201⁺) (STEMCELL Technologies Inc, Vancouver, British Columbia, Canada) were seeded onto 96-well microplate in X-VIVO 15 medium. The PBMCs were treated with Peptide 1 (SEQ ID NO: 1) or Peptide 12 (SEQ ID NO: 12) of the present invention, which was serially diluted from 250 nM to 0.4 nM in 5-fold. The culture medium was refreshed every 5 days, and on the 12th day after treatment with the peptides, the cells were collected and stained with anti-CD4-PerCP-Cy5.5 conjugated, IFN-γ-FITC chrome conjugated, and IL4-PE chrome conjugated antibodies according to the manufacturer's instruction. After that, the cells were washed twice in cold FACS buffer, resuspended in FACS buffer, and analyzed in a flow cytometer ((BD LSRFortessa™X20, Franklin Lakes, N.J., U.S.).

Results

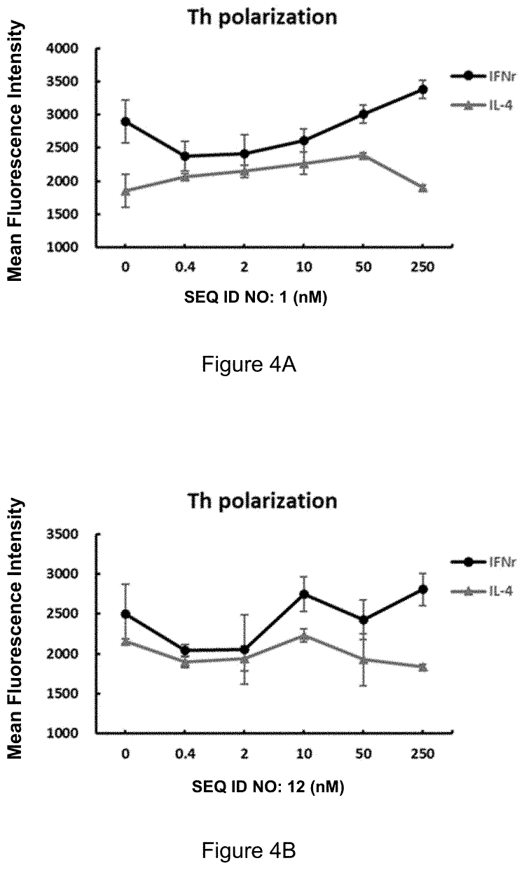

The peptides of the present invention stimulate immune cells to secret cytokines enhancing both cellular and humoral immunities. As shown in FIG. 4A, Peptide 1 (SEQ ID NO: 1) of the present invention stimulated T helper cells (CD4⁺ cells) to secret higher level of IFN-γ at a concentration of 250 nM and of IL-4 at a concentration of 50 nM. As shown in FIG. 4B, Peptide 12 (SEQ ID NO: 12) of the present invention stimulated CD4⁺ cells to secret higher level of IFN-γ at the concentrations of 10 nM and 250 nM and of IL-4 at a concentration of 10 nM.

The results indicate that the peptides of the present invention are able to stimulate T helper cells to secret the cytokines IFN-γ and IL-4, which represent the cellular and humoral immunity, respectively. In addition, by stimulating T helper cells to secret IFN-γ, the peptides of the present invention enhance CTL immune response, which corresponds to the results of Example 2.

### Example 4 Immunogenicity Assay of Peptide 1 by Intramuscular Injection

### Materials and Methods

Immunization of mice. To determine the immunogenicity of the antigen peptides of the present invention, Peptide 1 (SEQ ID NO: 1) of the present invention were used as an example for mice model. Ten (10) female BALB/c mice (7-9 weeks old) supplied by Envigo (Indianapolis, Ind., USA) were intramuscularly injected with 3 shots of 45 μg Peptide 1 (SEQ ID NO: 1) at 2 weeks apart (on Days 0, 14, and 28). Serum was collected before the day of each immunization (on Days−1, 13, and 27). Terminal blood collection was performed on Day 41. Isolated serum was stored at −80° C. until serological analysis. Serum samples collected one day prior to the first injection (on Day−1, pre-dose group) and serum samples collected 2 weeks after the third injection (on Day 41, post-dose group) were used for anti-spike IgG ELISA assay.

Total IgG ELISA. IgG (Total) Mouse Uncoated ELISA kit (Cat. No. 88-50400, Thermo Fisher Scientific, MA, USA) was used for total IgG ELISA according to manufacturer's instruction. Briefly, Nunc™ MaxiSorp™ 9018 ELISA plate was coated with 100 μL/well of capture antibody in Coating Buffer and incubated overnight at 4° C. The plate was washed twice with 400 μL/well Wash Buffer. After the wells were blocked with 250 μL of Blocking Buffer and incubated at room temperature for 2 hours, the plate was washed twice. The standards were serially diluted with Assay Buffer A at 2-fold to make the standard curve. Then, 100 μL/well of Assay Buffer A were added to the blank wells, and 90 μL/well of Assay Buffer A were added to the sample wells. Serum samples were diluted at least 10,000-fold in Assay Buffer A, and then 10 μL/well of the prediluted serum samples were added to the appropriate wells. Fifty (50) μL/well of diluted Detection Antibody were added to all wells. Then, the plate was covered and incubated at room temperature for 2 hours, and washed four times after the incubation. One-hundred (100) μL/well of Substrate Solution were added to each well, and the plate was incubated at room temperature for 15 minutes. After that, 100 μL of Stop Solution were added to each well, and the plate was read at OD 450 nm with a microplate reader.

Anti-spike IgG ELISA. Human SARS-CoV-2 Spike (trimer) IgG ELISA kit (Cat. No. BMS2325, Thermo Fisher Scientific, MA, USA) was used for anti-spike IgG ELISA according to manufacturer's instruction. Briefly, a Human SARS-CoV-2 Spike (trimer) Coated Plate (96 wells) was washed with Wash Buffer, and then 90 μL of Assay Buffer and 10 μL of Assay Buffer diluted sample were added to a well of the plate. The plate was covered with a plate cover and incubated at 37° C. for 30 minutes. After incubation, the plate was washed with Wash Buffer, and 100 μL of anti-mouse horseradish peroxidase (HRP)-conjugated detection antibody (A90-131P, Bethyl, Tex., USA) were added to a well of the plate. The plate was covered with the plate cover and incubated at 37° C. for another 30 minutes. After incubation, the plate was washed with Wash Buffer, and 100 μL of Substrate Solution (Tetramethylbenzidine, TMB) were added to a well of the plate. The plate was incubated at room temperature for 15 minutes. After incubation, 100 μL of Stop Solution were added to a well of the plate. Then, the plate was read at OD 450 nm with a microplate reader.

Statistical analysis. Prism 5 (GraphPad Software Inc., San Diego, Calif., USA) was used for statistical analysis. t-test was used to calculate significance. * p<0.05, ** p<0.01, *** p<0.001.

Results

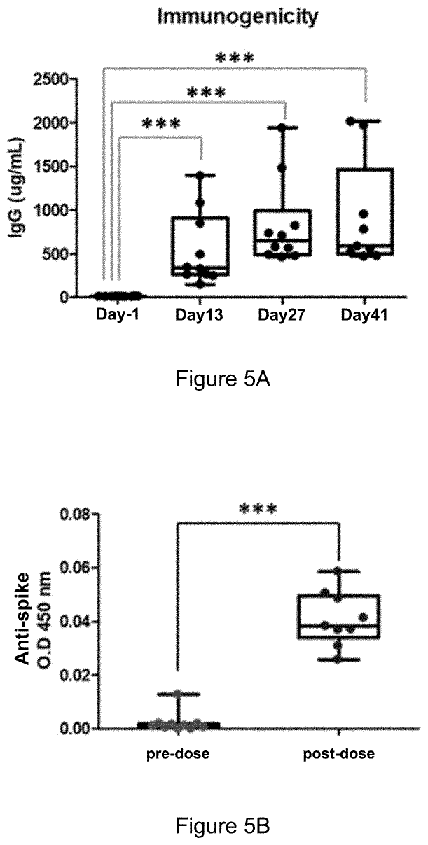

Intramuscular administration of the peptides of the present invention induces immunogenicity against SARS-CoV-2 spike protein. As shown in FIG. 5A, intramuscular administration of Peptide 1 (SEQ ID NO: 1) of the present invention to mice induced total IgG antibodies, in which the efficacy reaches to the plateau after second administration. The results indicate that the peptides of the present invention have immunogenicity.

As shown in FIG. 5B, intramuscular administration of Peptide 1 (SEQ ID NO: 1) of the present invention to mice induced anti-spike IgG antibodies. The results further indicate that the peptides of the present invention induce mice to produce specific antibodies against human SARS-CoV-2 spike protein.

### Example 5 Immunogenicity Assay of Peptide 12 by Intramuscular Injection

Materials and Methods

Immunization of mice. To determine the immunogenicity of the antigen peptides of the present invention, Peptide 12 (SEQ ID NO: 12) of the present invention were used as examples for mice model. Four (4) female BALB/c mice (7-9 weeks old) supplied by BioLASCO (Taiwan Co. Ltd) were intramuscularly injected with 3 shots of 400 μg Peptide 12 (SEQ ID NO: 12) at 2 weeks apart (on Days 0, 14, and 28). Serum were collected one day prior to the first injection (on Day−1, pre-dose group) and 2 weeks after the third injection (on Day 41, post-dose group). Isolated serum was stored at −80° C. until serological analysis.

Total IgG ELISA. Total IgG ELISA assay was performed as described in Example 4.

Anti-spike IgG ELISA. Anti-spike IgG ELISA assay was performed as described in Example 4.

Statistical analysis. Statistical analysis was performed as described in Example 4.

Results

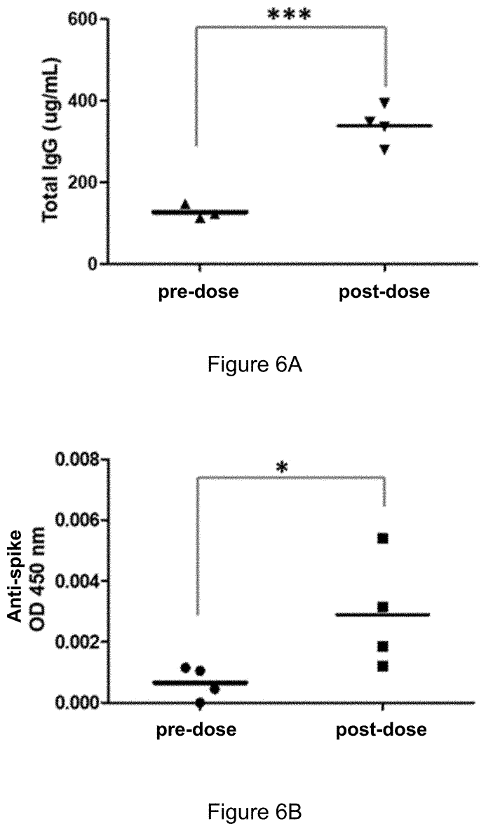

Intramuscular administration of the peptides of the present invention induces immunogenicity against SARS-CoV-2 spike protein. As shown in FIG. 6A, intramuscular administration of Peptide 12 (SEQ ID NO: 12) of the present invention to mice induced total IgG antibodies. The results indicate that the peptides of the present invention have immunogenicity. The results of this Example also correspond to the results of Example 2, in which the peptides of the present invention stimulate T helper cells to secret IL-4, which activates B cells to produce antibodies.

As shown in FIG. 6B, intramuscular administration of Peptide 12 (SEQ ID NO: 12) of the present invention to mice induced anti-spike IgG antibodies. The results further indicate that the peptides of the present invention induce mice to produce specific antibodies against human SARS-CoV-2 spike protein.

### Example 6 Immunogenicity Assay of Peptide 1 by Oral Administration

Materials and Methods

Immunization of mice. To determine the immunogenicity of the antigen peptides of the present invention, Peptide 1 (SEQ ID NO: 1) of the present invention were used as examples for mice model. Five (5) female BALB/c mice (7-9 weeks old) supplied by BioLASCO (Taiwan Co. Ltd) were oral administrated with 3 doses of 200 μL of 1 μg/μL Peptide 1 (SEQ ID NO: 1; 200 μg per dose) formulated with 1% (v/v) of 77 mg/mL PLCL-PEG-PLCL (Sigma-Aldrich, St. Louis, Mo., USA) at 2 weeks apart (on Days 0, 14, and 28). Serum were collected one day prior to the first dose (on Day−1, pre-dose group) and 2 weeks after the third dose (on Day 41, post-dose group). Isolated serum was stored at −80° C. until serological analysis.

Total IgG ELISA. Total IgG ELISA assay was performed as described in Example 4.

Anti-spike IgG ELISA. Anti-spike IgG ELISA assay was performed as described in Example 4.

Statistical analysis. Statistical analysis was performed as described in Example 4.

Results

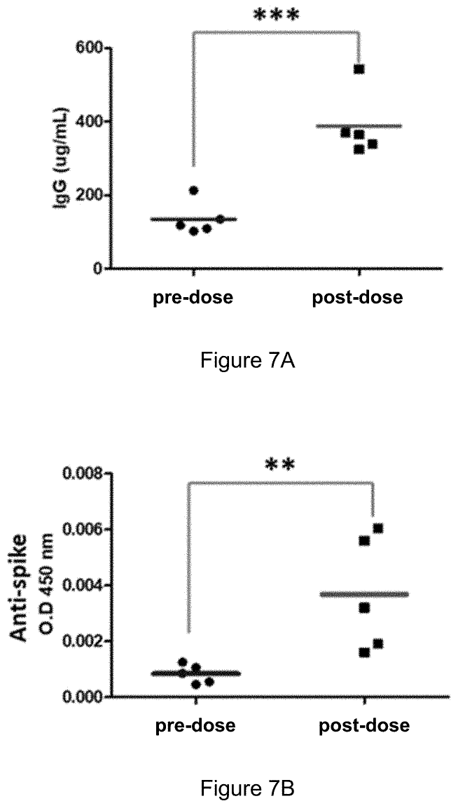

Oral administration of the peptides of the present invention induces immunogenicity against SARS-CoV-2 spike protein. As shown in FIG. 7A, oral administration of Peptide 1 (SEQ ID NO: 1) of the present invention to mice induced total IgG antibodies. The results indicate that the peptides of the present invention have immunogenicity.

As shown in FIG. 7B, oral administration of Peptide 1 (SEQ ID NO: 1) of the present invention to mice induced anti-spike IgG antibodies. The results further indicate that the peptides of the present invention induce mice to produce specific antibodies against human SARS-CoV-2 spike protein.

The peptides of the present invention are designed specifically for binding to human MHC molecules, and therefore, these peptides have high affinity to HLA-A201 (as shown in FIG. 2). However, the peptides of the present invention also have lower affinity to mouse MHC molecules. For example, it is estimated that Peptide 1 (SEQ ID NO: 1) of the present invention has the half maximal inhibitory concentration (IC50) of 2.2, 78.1, and 82.5 μM to mouse MHC class H2-Kd, H2-Ld and H-2-Dd molecules, respectively (https://www.iedb.org). With the lower affinity to mouse MHC molecules, the peptides of the present invention are still able to induce immune response in mice (as shown in FIGS. 5A to 7B). These results indicate that the peptides of the present invention are able to evoke not only immune response with different subtype of MHC molecules, but also higher immune response in human subjects.

In conclusion, the peptides of the present invention exhibit strong binding affinities to MHC molecules, especially to MHC class I molecules, induce both CD4⁺ and CD8⁺ T cell responses, engage in CTL response, and stimulate the production of specific antibodies against human SARS-CoV-2 spike protein via both intramuscular and oral routes. Since the peptides of the present invention were designed based on the well-conserved regions of human SARS-CoV-2 spike protein and induce both cellular and humoral immune responses, the peptides are great candidates for development of broad-spectrum vaccine against coronavirus, especially SARS-CoV-2.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A peptide, comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, and wherein the variant binds to a molecule of a major histocompatibility complex (MHC) and/or induces T cells cross-reacting with the variant peptide.

2. The peptide according to claim 1, wherein the peptide has the ability to bind to an MHC class-I or II molecule, and wherein the peptide, when bound to the MHC, is capable of being recognized by CD4⁺ and/or CD8⁺ T cells.

3. A nucleic acid encoding the peptide according to claim 1.

4. An immunogenic composition, comprising a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, or a nucleic acid according to claim 3, wherein the variant binds to a molecule of a major histocompatibility complex (MHC) and/or induces T cells cross-reacting with the variant peptide.

5. The immunogenic composition according to claim 4, further comprising a pharmaceutically acceptable carrier and/or an adjuvant.

6. An antibody specifically recognizing the peptide according to claim 1.

7. A T-cell receptor being capable of binding to the peptide according to claim 1.

8. The T-cell receptor according to claim 7, wherein the peptide is bound to an MHC molecule.

9. A recombinant host cell, comprising one component selected from the group consisting of a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, a nucleic acid encoding the peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, an antibody or fragment thereof specifically recognizing the peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, and a T-cell receptor or fragment thereof being capable of binding to the peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, wherein the variant binds to a molecule of a major histocompatibility complex (MHC) and/or induces T cells cross-reacting with the variant peptide.

10. The recombinant host cell according to claim 9, wherein the host cell is selected from a dendritic cell, a T cell, or a natural killer (NK) cell.

11. An in vitro or ex vivo method for producing activated T lymphocytes, comprising contacting in vitro or ex vivo T cells with antigen loaded human class I or II MHC molecules expressed on the surface of a suitable antigen-presenting cell or an artificial construct mimicking an antigen-presenting cell for a period of time sufficient to activate the T cells in an antigen specific manner, wherein the antigen is the peptide according to claim 1.

12. An activated T lymphocyte produced by the method according to claim 11, wherein the activated T lymphocyte selectively recognizes a cell presenting a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, and wherein the variant binds to a molecule of a major histocompatibility complex (MHC) and/or induces T cells cross-reacting with the variant peptide.

13. A pharmaceutical composition, comprising at least one active ingredient selected from the group consisting of a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, a nucleic acid encoding the peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, an antibody or fragment thereof specifically recognizing the peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, a T-cell receptor or fragment thereof being capable of binding to the peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, the recombinant host cell according to claim 9, and an activated T lymphocyte selectively recognizing a cell presenting the peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26, wherein the variant binds to a molecule of a major histocompatibility complex (MHC) and/or induces T cells cross-reacting with the variant peptide.

14. The pharmaceutical composition according to claim 13, further comprising a pharmaceutically acceptable carrier, and/or pharmaceutically acceptable excipients and/or stabilizers.

15. A method for preventing or treating a pathogenic infection in a subject in need thereof, comprising administering to the subject an effective amount of the pharmaceutical composition according to claim 13.

16. The method according to claim 15, wherein the pathogenic infection is induced by a coronavirus.

17. The method according to claim 16, wherein the coronavirus is the severe acute respiratory syndrome coronavirus 2 (SARS-CoV-2).

18. A method for generating anti-coronavirus antibodies, comprising administering the immunogenic composition according to claim 4 to an animal or a human subject.

19. The method of claim 18, wherein the anti-coronavirus antibodies are characterized by having binding affinity to a coronavirus.

20. The method of claim 19, wherein the coronavirus is the severe acute respiratory syndrome coronavirus 2 (SARS-CoV-2).

21. A complex, comprising a peptide bound to an MHC molecule, wherein the peptide comprises an amino acid sequence selected from the group consisting of SEQ ID NO: 1 to SEQ ID NO: 26 and variant sequences thereof which are at least 80% homologous to SEQ ID NO: 1 to SEQ ID NO: 26, and wherein the variant binds to a molecule of a major histocompatibility complex (MHC) and/or induces T cells cross-reacting with the variant peptide.

22. The complex according to claim 21, wherein the complex comprises two or more of the peptides and two or more of the MHC molecules.

23. The complex according to claim 22, wherein each of the two or more of the peptides is bound to one of the two or more of the MHC molecules.

24. The complex according to claim 22, wherein each of the two or more of the MHC molecules is attached to a dextran backbone.

25. The complex according to claim 24, wherein the complex further comprises a fluorophore, optionally wherein the fluorophore is attached to the dextran backbone.

26. A method for determining the presence or absence of current or previous coronavirus infection in an individual, comprising contacting a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26 or the complex according to claim 21 with a sample obtained from the individual and determining the presence or absence of binding between the peptide or complex and a molecule comprised in the sample, wherein the variant binds to a molecule of a major histocompatibility complex (MHC) and/or induces T cells cross-reacting with the variant peptide.

27. The method according to claim 26, wherein the molecule is an antibody or a T cell receptor.

28. The method according to claim 26, wherein the presence of binding indicates the presence of current or previous coronavirus infection, and/or the absence of binding indicates the absence of current or previous coronavirus infection.

29. A method for identifying coronavirus-specific T cells, comprising contacting a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26 or the complex according to claim 21 with a sample obtained from an individual and determining the presence or absence of binding between the peptide or complex and a T cell receptor comprised in the sample, wherein the variant binds to a molecule of a major histocompatibility complex (MHC) and/or induces T cells cross-reacting with the variant peptide.

30. The method according to claim 29, wherein the individual is currently infected with the coronavirus.

31. The method according to claim 29, wherein the individual was previously, but is not currently, infected with the coronavirus.

32. A method for identifying a coronavirus-specific T cell receptor, comprising contacting a peptide comprising an amino acid sequence selected from the group consisting of SEQ ID NO: 1 to SEQ ID NO: 26 and variant sequences thereof which are at least 80% homologous to SEQ ID NO: 1 to SEQ ID NO: 26 or the complex according to claim 21 with a T cell receptor and determining the presence or absence of binding between the peptide or complex and the T cell receptor, wherein the variant binds to a molecule of a major histocompatibility complex (MHC) and/or induces T cells cross-reacting with the variant peptide.

33. The method according to claim 32, wherein the presence of binding indicates that the T cell receptor is a coronavirus-specific T cell receptor, and/or the absence of binding indicates that the T cell receptor is not coronavirus-specific T cell receptor.


SEQUENCE LISTING (27 sequences)

SEQ ID NO: 1 - protein, 9 aa, Artificial Sequence
  MHC binding peptide
KLNSGDSKV

SEQ ID NO: 2 - protein, 9 aa, Artificial Sequence
  MHC binding peptide
KMNSGDSKV

SEQ ID NO: 3 - protein, 9 aa, Artificial Sequence
  MHC binding peptide
KINSGDSKV

SEQ ID NO: 4 - protein, 9 aa, Artificial Sequence
  MHC binding peptide
KQNSGDSKV

SEQ ID NO: 5 - protein, 9 aa, Artificial Sequence
  MHC binding peptide
KVNSGDSKV

SEQ ID NO: 6 - protein, 9 aa, Artificial Sequence
  MHC binding peptide
KLNSGDSFV

SEQ ID NO: 7 - protein, 9 aa, Artificial Sequence
  MHC binding peptide
KLNSGDSKL

SEQ ID NO: 8 - protein, 9 aa, Artificial Sequence
  MHC binding peptide
KLNSGDSKI

SEQ ID NO: 9 - protein, 9 aa, Artificial Sequence
  MHC binding peptide
KLNSGDSKA

SEQ ID NO: 10 - protein, 9 aa, Artificial Sequence
  MHC binding peptide
KLNSGDSPV

SEQ ID NO: 11 - protein, 9 aa, Artificial Sequence
  MHC binding peptide
KLNSGISKV

SEQ ID NO: 12 - protein, 9 aa, Artificial Sequence
  MHC binding peptide
KLNSGLSKV

SEQ ID NO: 13 - protein, 9 aa, Artificial Sequence
  MHC binding peptide
KLNSGVSKV

SEQ ID NO: 14 - protein, 9 aa, Artificial Sequence
  MHC binding peptide
KLMSGDSKV

SEQ ID NO: 15 - protein, 9 aa, Artificial Sequence
  MHC binding peptide
KLWSGDSKV

SEQ ID NO: 16 - protein, 9 aa, Artificial Sequence
  MHC binding peptide
KLFSGDSKV

SEQ ID NO: 17 - protein, 9 aa, Artificial Sequence
  MHC binding peptide
KLYSGDSKV

SEQ ID NO: 18 - protein, 9 aa, Artificial Sequence
  MHC binding peptide
FLNSGDSKV

SEQ ID NO: 19 - protein, 9 aa, Artificial Sequence
  MHC binding peptide
YLNSGDSKV

SEQ ID NO: 20 - protein, 9 aa, Artificial Sequence
  MHC binding peptide
KLNSGDWKV

SEQ ID NO: 21 - protein, 9 aa, Artificial Sequence
  MHC binding peptide
KLNSGDFKV

SEQ ID NO: 22 - protein, 9 aa, Artificial Sequence
  MHC binding peptide
KLNSGDYKV

SEQ ID NO: 23 - protein, 9 aa, Artificial Sequence
  MHC binding peptide
KLNSGDSYV

SEQ ID NO: 24 - protein, 10 aa, Artificial Sequence
  MHC binding peptide
KLNSGDSKVV

SEQ ID NO: 25 - protein, 10 aa, Artificial Sequence
  MHC binding peptide
KLNSGDSKVL

SEQ ID NO: 26 - protein, 10 aa, Artificial Sequence
  MHC binding peptide
FKLNSGDSKV

SEQ ID NO: 27 - protein, 9 aa, Artificial Sequence
  Cytomegalovirus (CMV) pp65 protein-derived peptide
NLVPMVATV